(19) United States
(12) Patent Application Publication
(10) Pub. No.: US 2002/0103379 A1
(43) Pub. Date: Aug. 1, 2002

(54) Process for making a thrombin inhibitor

(76) Inventors: Ulf H. Dolling, Westfield, NJ (US); Ramon Alabaster, Puckeridge (GB); Michael S. Ashwood, Bishops Stortford (GB); Ian F. Cottrell, Hertford (GB); Cameron J. Cowden, Stanstead Abbotts (GB); Antony J. Davies, Hertford (GB); David Hands, London (GB); Debra J. Wallace, Stanstead Abbotts (GB); Robert D. Wilson, London (GB)

Correspondence Address: MERCK AND CO INC, P O BOX 2000, RAHWAY, NJ, 070650907

(21) Appl. No.: 10/013,063

(22) Filed: Dec. 6, 2001

(30) Foreign Application Priority Data
Dec. 6, 2000 (GB) 0029728.3
Mar. 9, 2001 (GB) 0105911.2

(51) Int. Cl. C07D 213/28

(52) U.S. Cl. 546/339

(57) ABSTRACT

The invention is a process for preparing 3-fluoro-2-pyridylmethyl-3-(2,2-difluoro-2-(2-pyridyl)ethylamino)-6-chloropyrazin-2-one-1-acetamide which comprises coupling

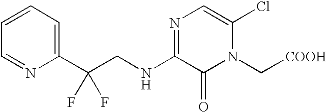

with 2-aminomethyl-3-fluoropyridine dihydrochloric acid salt in the presence of a coupling reagent and a base to form 3-fluoro-2-pyridylmethyl-3-(2,2-difluoro-2-(2-pyridyl)ethylamino)-6-chloropyrazin-2-one-1-acetamide.

### BACKGROUND OF THE INVENTION

Taguchi, et al., Tetrahedron Letters, vol. 27, no. 50, pp. 6103-6106 (1986) describe a method for preparing 2,2-difluoroesters by reacting activated iododifluoroacetate-copper with organic halides. Sato, et al. Chem. Pharm. Bull. Vol. 47 no. 7 pp. 1013-1016 (1999) describe a method for preparing alkenyl- and aryldifluoroacetate from ethyl bromodifluoroacetate and halogenated compounds using copper powder. Sato, et al. Chem. Pharm. Bull. Vol. 48 no. 7 pp. 1023-1025 (2000) describe reaction of ethyl bromodifluoroacetate with α,β-unsaturated carbonyl compounds in the presence of copper powder. Cockerill, et al. J. Chem. Soc., Perkin Trans. I 2000, 2591-2599 describe coupling of 1,4-diidobenzene with the organozinc reagent derived from diethyl bromodifluoromethylphosphonate with copper catalysis.

Fleitz, et al. Synthetic Communications vol. 30 no. 17 pp. 3171-3180 (2000) describe synthesis of ethyl 3-(2,2-difluoro-2-(2-pyridylethylamino)pyrazin(1H)-2-one-1-acetate from 3,4-dihydro-6-methyl-2,3-dioxo-1(2H)-pyrazineacetic acid ethyl ester.

The present invention provides an efficient process for preparing the thrombin inhibitor 3-fluoro-2-pyridylmethyl-3-(2,2-difluoro-2-(2-pyridyl)ethylamino)-6-chloropyrazin-2-one-1-acetamide and related compounds.

### SUMMARY OF THE INVENTION

The invention comprises processes for making 2,2-difluoro-2-(2-pyridyl)-ethan-1-ol, ethyl 3-chloropyrazin(1H)-2-one-acetate and 2-aminomethyl-3-fluoropyridine, and coupling of these, and further transformation, to give 3-fluoro-2-pyridylmethyl-3-(2,2-difluoro-2-(2-pyridyl)ethylamino)-6-chloropyrazin-2-one-1-acetamide or related compounds.

### DETAILED DESCRIPTION OF THE INVENTION

The invention is a process for preparing 2,2-difluoro-2-(2-pyridyl)-ethan-1-ol, which comprises coupling 2-bromopyridine with ethyl 2-bromo-2,2-difluoroacetate, in the presence of metallic copper, preferably a non-active copper powder, and a solvent, e.g. a non-reactive solvent such as dimethyl sulfoxide or, more preferably, dimethylformamide, to form 2,2-difluoro-2-(2-pyridyl)acetate.

The invention is also a process for reducing 2,2-difluoro-2-(2-pyridyl)acetate with a reducing agent, e.g., a borohydride or aluminium hydride reagent such as sodium borohydride, lithium borohydride, sodium aluminium hydride, or lithium aluminium hydride, in the presence of a solvent, e.g. an alcohol such as ethanol, to form the 2,2-difluoro-2-(2-pyridyl)-ethan-1-ol.

The invention also includes a process for preparing 2,2-difluoro-2-(2-pyridyl)ethylamine benzenesulfonate salt which comprises adding trifluoromethanesulfonic anhydride to a solution of 2,2-difluoro-2-(pyridyl)ethanol in acetonitrile, and thereafter adding an ammonium reagent such as aqueous ammonia and a solution of benzenesulfonic acid to form 2,2-difluoro-2-(2-pyridyl)ethylamine benzenesulfonate salt.

The invention also includes a process for preparing ethyl 2-{(N-(2,2-dimethoxyethyl)carbamoyl}carbonylamino)acetate which comprises reacting ethyl glycine hydrochloric acid salt with ethyl oxalyl chloride in the presence of a first non-reactive solvent, e.g. isopropyl acetate, to form a reaction product, adding 2,2-dimethoxyethylamine to the reaction product to form ethyl 2-{(N-(2,2-dimethoxyethyl)carbamoyl}carbonylamino)acetate, and precipitating the ethyl 2-{(N-(2,2-dimethoxyethyl)carbamoyl}carbonylamino) acetate with a second non-reactive solvent, e.g. heptane.

The invention also includes a process for preparing ethyl 2-(2,3-dioxo-1,4-dihydropyrazinyl) acetate which comprises cyclizing ethyl 2-{(N-(2,2-dimethoxyethyl)carbamoyl}carbonylamino)acetate in the presence of an acid reagent such as trifluoroacetic acid in a solvent such as acetic acid.

The invention also includes a process for preparing ethyl 3-chloropyrazin(1H)-2-one-1-acetate which comprises reacting ethyl 2-(2,3-dioxo-1,4-dihydropyrazinyl)acetate with a chlorinating reagent, e.g. thionyl chloride, oxalyl chloride, phosphorous oxychloride, phosphorous pentachloride, a mixture of triphenylphosphine and N-chlorosuccinimide, and a mixture of tetrabutyl ammonium chloride and phosphorous pentoxide, in the presence of a non-reactive solvent, e.g. isopropyl acetate, 1,2-dichloroethane, toluene, dimethylformamide, ethyl acetate, dichloromethane, chloroform, carbon tetrachloride, benzene, propionitrile, acetonitrile and methyl acetate, to form ethyl 3-chloropyrazin(1H)-2-one-1-acetate.

The invention also includes a process for preparing ethyl 3-(2,2-difluoro-2-(2-pyridylethylamino)pyrazin(1H)-2-one-1-acetate which comprises coupling ethyl 3-chloropyrazin(1H)-2-one-1-acetate with 2,2-difluoro-2-(2-pyridyl)ethylamine benzenesulfonate salt in the presence of a base, such as N,N diisopropylethylamine, triethyl amine, or tributyl amine, a non-reactive solvent such as acetonitrile, and an iodide, such as sodium iodide, to form ethyl 3-(2,2-difluoro-2-(2-pyridylethylamino)pyrazin(1H)-2-one-1-acetate.

The invention is also a process for preparing 3-fluoro-2-pyridylmethyl-3-(2,2-difluoro-2-(2-pyridyl)ethylamino)-6-chloropyrazin-2-one-1-acetamide which comprises coupling

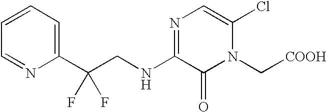

with 2-aminomethyl-3-fluoropyridine dihydrochloric acid salt in the presence of a coupling reagent such as 1-(3-dimethylaminopropyl)-3-ethylcarbo-diimide hydrochloride or 1,3-dicyclocarbdiimide, optionally additionally including 1-hydroxybenzotriazole hydrate or 1-hydroxy-7-azabenzotriazole, and a base such as potassium hydroxide, to form 3-fluoro-2-pyridylmethyl-3-(2,2-difluoro-2-(2-pyridyl)ethylamino)-6-chloropyrazin-2-one-1-acetamide.

The process includes, for example, preparing 3-fluoro-2-acetamide by coupling ethyl 3-(2,2-difluoro-2-(2-pyridylethylamino)pyrazin(1H)-2-one-1-acetate with 2-aminomethyl-3-fluoropyridine dihydrochloric acid salt in the presence of a base such as potassium hydroxide to form 3-fluoro-2-pyridylmethyl-3-(2,2-difluoro-2-(2-pyridyl)ethylamino)-6-chloropyrazin-2-one-1-acetamide.

The invention also includes a compound having the formula

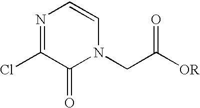

wherein R is C₁₋₄ alkyl, e.g. —CH₂CH₃.

The invention also includes a process for recrystallizing 2-aminomethyl-3-fluoropyridine dihydrochloride which comprises suspending 2-aminomethyl-3-fluoropyridine dihydrochloride in acetic acid, heating to dissolve the 2-aminomethyl-3-fluoropyridine dihydrochloride into a solution, and precipitating the 2-aminomethyl-3-fluoropyridine dihydrochloride from the solution.

The invention also includes a process for preparing 2-{3-[(2,2-difluoro-2-(2-pyridyl)ethyl)amino]-6-chloro-2-oxohydro-pyrazinyl}acetate which comprises chlorinating ethyl 3-(2,2-difluoro-2-(2-pyridylethylamino)pyrazin(1H)-2-one-1-acetate with N-chlorosuccinimide or 1,3 dichloro-5,5-dimethylhydantoin in the presence of a non-reactive solvent such as acetonitrile.

The invention also includes a process for preparing 2-cyano-3-fluoropyridine comprising adding chlorotrimethylsilane to a mixture comprising 3-fluoropyridine N-oxide and potassium cyanide to form 2-cyano-3-fluoropyridine.

The invention also includes a process for preparing ethyl 2-(2,3-dioxo-1,4-dihydropyrazinyl)acetate which comprises preparing ethyl 2-{(N-(2,2-dimethoxyethyl)carbamoyl}carbonylamino) acetate by reacting ethyl glycine hydrochloric acid salt with ethyl oxalyl chloride in the presence of a first non-reactive solvent such isopropyl acetate to form a first reaction product, adding 2,2-dimethoxyethylamine to the first reaction product to form a second reaction product comprising ethyl 2-{(N-(2,2-dimethoxyethyl)carbamoyl}carbonylamino)acetate, and cyclizing ethyl 2-{(N-(2,2-dimethoxyethyl)carbamoyl}carbonylamino)acetate in the presence of an acid reagent such as trifluoroacetate and a second solvent such as acetic acid to form ethyl 2-(2,3-dioxo-1,4-dihydropyrazinyl)acetate.

The abbreviations listed below are used in the specification and have the meanings indicated below:

| | |
|---|---|
| BSA | benzenesulfonic acid |
| CH₃CN | acetonitrile |
| CH₃CO₂H | acetic acid |
| DMF | dimethylformamide |
| DMSO | dimethylsulfoxide |
| EDC | 1-(3-dimethylaminopropyl)-3-ethylcarbodiimide hydrochloride |
| EtOH | ethanol |
| Et₃N | triethylamine |
| HOBt | 1-hydroxybenzotriazole hydrate |
| IPAc | isopropyl acetate |
| KCN | potassium cyanide |
| KHCO₃ | potassium hydrogen carbonate |
| KOH | potassium hydroxide |
| MeCN | acetonitrile |
| NaBH₄ | sodium borohydride |
| NaI | sodium iodide |
| Na₂CO₃ | sodium carbonate |
| NCS | N-chlorosuccinimide |
| NMP | N-methyl pyrrolidinone |
| s.g. | specific gravity |
| SOCl₂ | thionyl chloride |
| TFA | trifluoroacetic acid |
| Tf₂O | trifluoromethane sulfonic anhydride |
| THF | tetrahydrofuran |
| TMSCl | trimethylsilyl chloride |

Metallic copper useful in the process of the invention includes activated and non activated copper, including copper bars, copper foil, copper granules, copper powder, copper rods, copper shots, copper turnings, and copper wire. Preferably, the copper is a non-activated metallic copper powder. Suitable non-activated copper powders include dendritic 3 micron, −40 mesh, −150 mesh, submicron, copper bronze, −200 mesh, and spheroidal powders.

The term “alkyl” includes branched or straight chain alkyl groups. “C₁₋₄ alkyl” refers to alkyl groups having 1, 2, 3, or 4 carbon atoms, e.g. methyl, ethyl, propyl, isopropyl, etc.

Pharmaceutically-acceptable salts of compounds prepared according to the process of the invention (in the form of water- or oil-soluble or dispersible products) include the conventional non-toxic salts such as those derived from inorganic acids, e.g. hydrochloric, hydrobromic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

In order to determine activity of compounds prepared according to the process of the invention, assays of human α-thrombin and human trypsin were performed by the methods substantially as described in Thrombosis Research, Issue No. 70, page 173 (1993) by S. D. Lewis et al.

Activity assays were performed by diluting a stock solution of substrate at least tenfold to a final concentration ≦0.1 Kₘ into a solution containing enzyme or enzyme equilibrated with inhibitor. Times required to achieve equilibration between enzyme and inhibitor were determined in control experiments. Initial velocities of product formation in the absence (Vₒ) or presence of inhibitor (Vᵢ) were measured. Assuming competitive inhibition, and that unity is negligible compared to Kₘ/[S] and [I]/e (where [S], [I], and e respectively represent the total concentrations, of substrate, inhibitor and enzyme), the equilibrium constant (Kᵢ) for dissociation of the inhibitor from the enzyme can be obtained from the dependence of Vₒ/Vᵢ on [I] shown in the equation: Vₒ/Vᵢ=1+[I]/Kᵢ.

The activities shown by this assay indicate that the compounds are therapeutically useful for treating various conditions in patients suffering from unstable angina, refractory angina, myocardial infarction, transient ischemic attacks, atrial fibrillation, thrombotic stroke, embolic stroke, deep vein thrombosis, disseminated intravascular coagulation, and reocclusion or restenosis of recanalized vessels. The compounds are selective compounds, as evidenced by their inhibitory activity against human trypsin (represented by Ki), which is at least 1000 nM.

Anticoagulant therapy is indicated for the treatment and prevention of a variety of thrombotic conditions, particularly coronary artery and cerebrovascular disease. Those experienced in this field are readily aware of the circumstances requiring anticoagulant therapy. The term “patient” used herein is taken to mean mammals such as primates, including humans, sheep, horses, cattle, pigs, dogs, cats, rats, and mice.

Compounds prepared according to the process of the invention are useful for treating or preventing venous thromboembolism (e.g. obstruction or occlusion of a vein by a detached thrombus; obstruction or occlusion of a lung artery by a detached thrombus), cardiogenic thromboembolism (e.g. obstruction or occlusion of the heart by a detached thrombus), arterial thrombosis (e.g. formation of a thrombus within an artery that may cause infarction of tissue supplied by the artery), atherosclerosis (e.g. arteriosclerosis characterized by irregularly distributed lipid deposits) in mammals, and for lowering the propensity of devices that come into contact with blood to clot blood.

The thrombin inhibitors can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, or intramuscular form, all using forms well known to those of ordinary skill in the pharmaceutical arts. An effective but non-toxic amount of the compound desired can be employed as an anti-aggregation agent. For treating ocular build up of fibrin, the compounds may be administered intraocularly or topically as well as orally or parenterally.

The thrombin inhibitors can be administered in the form of a depot injection or implant preparation which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The dosage regimen utilizing the thrombin inhibitors is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the thrombin inhibitors, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-0.5 mg/kg/day (unless specificed otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/day. A suitably prepared medicament for once a day administration would thus contain between 0.8 mg and 2.4 g, preferably between 2 mg and 600 mg, more preferably between 8 mg and 200 mg, and most preferably 8 mg and 40 mg, e.g., 8 mg, 10 mg, 20 mg and 40 mg. Advantageously, the thrombin inhibitors may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg.

Intravenously, the patient would receive the active ingredient in quantities sufficient to deliver between 0.025-7.5 mg/kg/day, preferably 0.1-2.5 mg/kg/day, and more preferably 0.1-0.5 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/ml, e.g. 0.1 mg/ml, 0.3 mg/ml, and 0.6 mg/ml, and administered in amounts per day of between 0.01 ml/kg patient weight and 10.0 ml/kg patient weight, e.g. 0.1 ml/kg, 0.2 ml/kg, 0.5 ml/kg. In one example, an 80 kg patient, receiving 8 ml twice a day of an intravenous formulation having a concentration of active ingredient of 0.5 mg/ml, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily made by a person having ordinary skill in the art.

The compounds can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The thrombin inhibitors are typically administered as active ingredients in admixture with suitable pharmaceutical diluents, excipients or carriers (collectively referred to herein as “carrier” materials) suitably selected with respect to the intended form of administration, that is, oral tablets, capsules, elixers, syrups and the like, and consistent with convention pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch methyl cellulose, agar, bentonite, xanthan gum and the like.

Typical uncoated tablet cores suitable for administration of thrombin inhibitors are comprised of, but not limited to, the following amounts of standard ingredients:

| | General | Preferred | Most Preferred |
|---|---|---|---|
| Excipient | Range (%) | Range (%) | Range (%) |
| mannitol | 10-90 | 25-75 | 30-60 |
| microcrystalline | 10-90 | 25-75 | 30-60 |
| cellulose | | | |
| magnesium stearate | 0.1-5.0 | 0.1-2.5 | 0.5-1.5 |

Mannitol, microcrystalline cellulose and magnesium stearate may be substituted with alternative pharmaceutically acceptable excipients.

The thrombin inhibitors can also be co-administered with suitable anti-platelet agents, including, but not limited to, fibrinogen receptor antagonists (e.g. to treat or prevent unstable angina or to prevent reocclusion after angioplasty and restenosis), anticoagulants such as aspirin, thrombolytic agents such as plasminogen activators or streptokinase to achieve synergistic effects in the treatment of various vascular pathologies, or lipid lowering agents including antihypercholesterolemics (e.g. HMG CoA reductase inhibitors such as lovastatin or simvastatin, HMG CoA synthase inhibitors, etc.) to treat or prevent atherosclerosis. For example, patients suffering from coronary artery disease, and patients subjected to angioplasty procedures, would benefit from coadministration of fibrinogen receptor antagonists and thrombin inhibitors. Also, thrombin inhibitors enhance the efficiency of tissue plasminogen activator-mediated thrombolytic reperfusion. Thrombin inhibitors may be administered first following thrombus formation, and tissue plasminogen activator or other plasminogen activator is administered thereafter.

Typical doses of the thrombin inhibitors in combination with other suitable anti-platelet agents, anticoagulation agents, or thrombolytic agents may be the same as those doses of thrombin inhibitors administered without coadministration of additional anti-platelet agents, anticoagulation agents, or thrombolytic agents, or may be substantially less that those doses of thrombin inhibitors administered without coadministration of additional anti-platelet agents, anticoagulation agents, or thrombolytic agents, depending on a patient's therapeutic needs.

The following schemes are exemplary and should not be interpreted as limiting the scope of the invention as defined above.

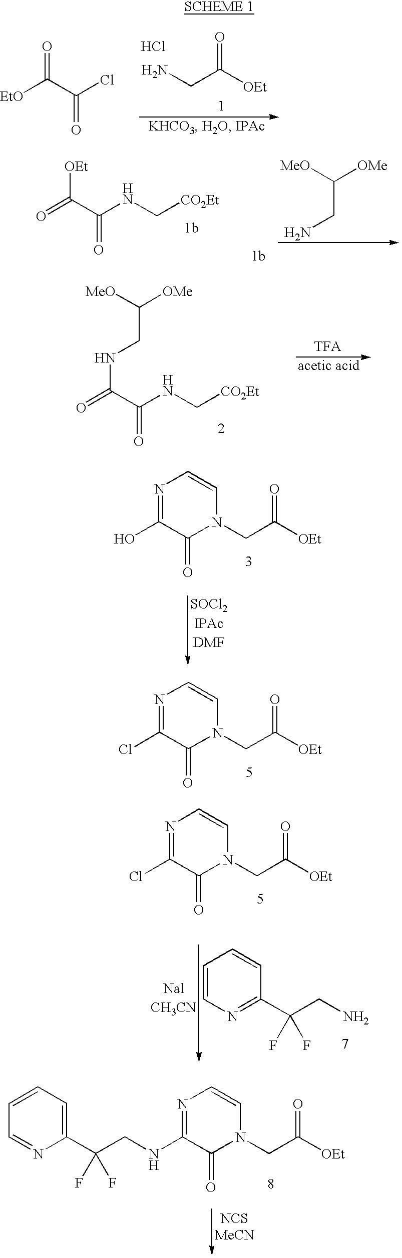

### Ethyl 2-{(N-(2,2-dimethoxyethyl)carbamoyl}carbonylamino)acetate (2)

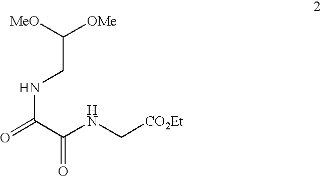

A mixture of ethyl glycine.HCl (1, 40.0 g, 286 mmol), potassium bicarbonate (172 g, 1.72 mol), water (120 mL) and isopropyl acetate (440 mL) was treated at 0° C. with ethyl oxalyl chloride (78.4 g, 574 mmol). More water (180 mL) was added and the organic phase was separated. The aqueous phase was re-extracted with isopropyl acetate (400 mL) and the combined organic phases concentrated to 300 mL volume. At 20° C., 2,2-dimethoxyethylamine (37.7 g, 358 mmol) was added and after 16 hours, the solution was concentrated to 100 mL and diluted with heptane (400 mL). The solid was collected and washed with heptane (90 mL) and dried to give crystalline oxamide 2 (65.7 g, 87% yield from 1).

Melting point: 63-65° C.

Proton NMR spectrum in methylene chloride d₂ at 400 MHz on Bruker DRX400 spectrometer. s=singlet, d=doublet, t=triplet, q=quartet, b=broad. 7.78 ppm, bt, 1H; 7.42 ppm bt, −5 Hz, 1H; 4.30 ppm, t, 5.27 Hz, 1H; 4.10 ppm, q, 7.14 Hz, 2H; 3.94 ppm, d, 5.91 Hz, 2H; 3.32 ppm, dd, 5.26 Hz, 6.22 Hz, 2H; 3.25 ppm, s, 6H; 1.15 ppm, t, 7.14 Hz, 3H.

### Ethyl 2-(2,3-dioxo-1,4-dihydropyrazinyl)acetate (3)

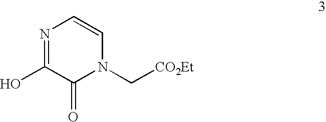

Trifluoroacetic acid (4.7 g, 41.5 mmol) was added to a mixture of ethyl 2-{(N-(2,2-dimethoxyethyl)carbamoyl}carbonylamino acetate 2 (10 g, 38.1 mmol) and acetic acid (25 mL). The stirred mixture was heated at 78° C. (internal temperature) and aged for 3 hours 45 minutes. The dark mixture was allowed to cool to 20° C. and stirred for 30 minutes before dilution with ethyl acetate (130 mL) and toluene (100 mL). After ageing at 20° C. for 30 minutes, the mixture was cooled to 5° C. and aged for a further 90 minutes. The product was collected by filtration, washed with ethyl acetate (50 ml), air dried and dried in vacuo at 45° C. to afford the title compound (3, 5.53 g, 73% yield) as a crystalline solid.

Melting point: 150-152° C.; crystalline by XRPD

Proton NMR spectrum in methanol d₄ at 400 MHZ on Bruker DRX400 spectrometer. s=singlet, d=doublet, t=triplet, q=quartet. 6.51 ppm, d, 5.96 Hz, 1H; 6.38 ppm, d, 5.96, 1H; 4.78 ppm, s, 2H; 4.22 ppm, q, 7.15 Hz, 2H; 1.28 ppm, t, 7.15 Hz, 3H.

### Ethyl 2-(2,3-dioxo-1,4-dihydropyrazinyl)acetate (3) (alternative process)

The following alternative procedure produces 3 directly from ethyl glycine hydrochloride 1. This process avoids the need for isolating ethyl 2-{(N-(2,2-dimethoxyethyl)carbamoyl}carbonylamino)acetate 2.

A mixture of potassium hydrogen carbonate (43 kg), water (15 L), isopropyl acetate (110 L) and ethyl glycine hydrochloride salt 1 (10 kg) were stirred together and cooled to 0° C. Ethyl oxalyl chloride (19.6 kg) was then added to the stirred mixture, at 0° C., over 2.3 h. More water (60 L) was added, the phases were separated and the aqueous phase was re-extracted with more isopropyl acetate (100 L). The combined isopropyl acetate extracts were concentrated to a volume of 130 L. and then 2,2-dimethoxyethylamine (9.43 kg) was added. The solution was stirred for 18 h.

The resulting solution of the oxamide 2 was then washed successively with a solution of citric acid (5 kg) in water (10 L), and then with a solution of potassium hydrogen carbonate (1.75 kg) in water (6.25 L). The resulting organic solution was then concentrated to a volume of 30 L and diluted with acetic acid (80 L). This solution was again concentrated at reduced pressure to a volume of 40 L. Acetic acid (15 L) and trifluoroacetic acid (5.1 L) were charged and the resulting solution heated to 78° C. for 3.5 h.

To the cooled solution was then added isopropyl acetate (214 L) followed by toluene (165 L). The slurry was stirred and cooled to 0-5° C. for 1 h and the solid collected by filtration. The solid was washed with toluene and dried to give 10.66 kg of the crystalline ester 3.

### Ethyl 3-chloropyrazin(1H)-2-one-1-acetate 5

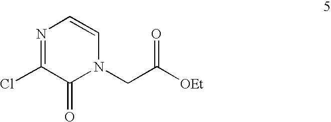

A mixture of ethyl 2-(2,3-dioxo-1,4-dihydropyrazinyl)acetate 3 (20 g, 100 mmol), isopropyl acetate (40 mL) and DMF (20 mL) was warmed to 45° C. To the mixture was added thionyl chloride (12.87 g) slowly over 10 minutes. The temperature was raised to 55-60° C. The chlorination was complete after 2 hrs. the reaction was diluted with isopropyl acetate (140 mL) and cooled to 30° C. 2M KHCO₃ (150 mL) was added slowly over 5 minutes. The two layers were separated, the aqueous phase was back-extracted with isopropyl acetate (90 mL). The combined organic phase was successively washed with 2 M KHCO₃ (50 mL) and water (2×50 mL). The organic phase was concentrated down to dryness in vacuo. The resulting yellow solid was flushed with tert-butyl methyl ether (2×20 mL). The solid was dried in vacuo at room temperature to afford the title compound 5 (19.4 g, 89% yield from 3).

Melting point: 47-49° C.; crystalline by XRPD

Proton spectrum in methylene chloride d₂, at 400 MHz on Bruker DRX 400, with tetramethylsilane as internal reference. d=doublet, t=triplet, s=singlet, q=quartet. 7.14 ppm, d, 4.4 Hz, 1H; 7.12 ppm, d, 4.4 Hz, 1H; 4.64 ppm, s, 2H; 4.24 ppm, q, 7.15 Hz, 2H; 1.28 ppm, t, 7.15 Hz,3H.

### Ethyl 3-chloropyrazin(1H)-2-one-1-acetate 5 (not isolated)

A mixture of ethyl 2-(2,3-dioxo-1,4-dihydropyrazinyl)acetate 3 (10 g, 50 mmol), isopropyl acetate (10 mL) and DMF (5 mL) was heated to 48-50° C. and a solution of thionyl chloride (6.6 g, 55.5 mmol) in isopropyl acetate (15 mL) added drop-wise. After the addition the temperature was increased to 55-60° C. The reaction initially formed a yellow-orange slurry, but within ten minutes a clear red solution was generated and the chlorination was complete after 1.5 hours. The mixture was allowed to cool to room temperature and isopropyl acetate (70 mL) was added before the reaction was quenched by the addition of KHCO₃ (75 mL of a 2 M aq sol). The two layers were separated, and the organic phase washed with KHCO₃ (32.5 mL), brine (32.5 mL) and water (6 mL). The organic phase was concentrated in vacuo to ˜15 mL and the solution flushed with acetonitrile (3×50 mL). Acetonitrile (24 mL) was added to give a crude solution of the ethyl 3-chloropyrazin(1H)-2-one-1-acetate 5 (9.7 g assay yield).

### Ethyl 3-(2,2-difluoro-2-(2-pyridylethylamino)pyrazin(1H)-2-one-1-acetate (8)

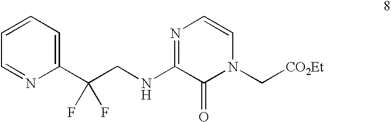

To the solution above was added the 2,2-difluoro-2-(2-pyridyl) benzenesulfonic acid 7 (the synthesis of which is described below)(14.28 g, 99.5 wt/wt %, 44.9 mmol), followed by N,N-diisopropylethylamine (5.8 g, 44.9 mmol) and sodium iodide (13.47 g, 89.8 mmol). The mixture was heated to reflux and aged for 24 hours and then cooled to room temperature. N.N-Diisopropylethylamine (5.8 g, 44.9 mmol) was added, followed by the slow addition of water (120 mL) during which crystallization of the product occurred. The slurry was cooled in an ice-bath at 0° C. for one hour and then filtered. After washing with water (3×40 mL) the product was dried in vacuo at 45° C. to afford the title compound 8 (12.76 g, 75% yield from 3) as crystalline needles.

Melting point: 92-94° C.; crystalline by XRPD

Proton NMR spectrum in methylene chloride d₂ at 400 MHz on Bruker DRX400 spectrometer. s=singlet, d=doublet, t=triplet, q=quartet, b=broad., 8.66 ppm, ddt, 0.87, 1.67, 4.77 Hz, 1H; 7.84 ppm, dt, 1.75, 7.79 Hz, 1H; 7.68 ppm, dt, 1.03, 7.87 Hz, 1H; 7.41 ppm, m, 1H; 6.81 ppm, d, 4.61 Hz, 1H; 6.50 ppm bt, 6.6 Hz, 1H; 6.44 ppm, d, 4.61 Hz, 1H; 4.52 ppm, s, 2H; 4.34 ppm, dt, 6.6, 14.4 Hz, 2H; 4.21 ppm, q, 7.07 Hz, 2H; 1.26 ppm, t, 7.07 Hz, 3H.

### Ethyl 2-{3-[(2,2-difluoro-2-(2-pyridyl)ethyl)amino]-6-chloro-2-oxohydro-pyrazinyl}acetate (9)

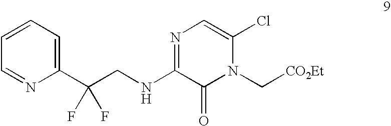

The ester 8 (20.0 g, 59.2 mmol) was slurried in acetonitrile (100 mL) and the mixture heated to 65° C. to give a solution. A solution of N-chlorosuccinimide (8.29 g, 62.1 mmol) in acetonitrile (100 mL) was then added over 10 minutes after which the reaction was maintained at 70° C. for 1 hour. The reaction mixture was cooled to 8° C. and water (450 mL) added drop-wise. The resulting mixture was aged at 5° C. for 1 hour and the solid collected by filtration. The product was washed with water (500 mL), air dried and dried in vacuo at 45° C. to give the title compound 9 (20.1 g, 91% yield) as a crystalline solid.

Melting point: 102-105° C. Crystalline by XRPD

Proton spectrum in methylene chloride d₂, at 250 MHz on Bruker DPX 250, with tetramethylsilane as internal reference. d=doublet, t=triplet, s=singlet, q=quartet. 8.65 ppm, d, 5.0 Hz, 1H; 7.85 ppm, dt, 1.9, 7.5 Hz, 1H; 7.68 ppm, dt, 1.25, 7.8 Hz, 1H; 7.40 ppm, m, 1H; 6.92 ppm, s, 1H; 6.55 ppm, bt, 6.3 Hz, 1H; 4.89 ppm, s, 2H; 4.34 ppm, dt, 6.6, 14.4 Hz, 2H; 4.22 ppm, q, 7.0 Hz, 2H; 1.25 ppm, t, 7.0 Hz, 3H.

### 3-Fluoro-2-pyridylmethyl-3-(2,2-difluoro-2-(2-pyridyl)ethylamino)-6-chloropyrazin-2-one-1-acetamide H₂O (12)

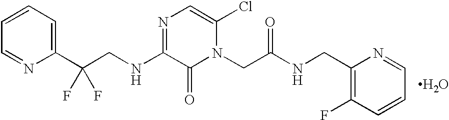

The chloroester 9 (3.15 kg, 8.45 mol) was dissolved in THF (85 L) and treated with potassium hydroxide (19.4 L of 1 M aq sol) after which the two-phase mixture was stirred at 40° C. for 4 hours. HOBt (1.14 kg, 8.45 mol) and amine.2HCl 11 (1.79 kg, 8.87 mol) were added followed by the slow addition of a solution of EDC.HCl (2.02 kg, 10.6 mol) in THF (10 L) over 1.75 hours with the reaction temperature maintained at 40° C. After a further 1 hour at this temperature, the reaction was allowed to cool to room temperature during which time crystallisation occurred. This was completed by addition of sodium bicarbonate (100 L of 3.5% aq sol). The product was collected by filtration, washed well with water (20 L), air dried and dried in vacuo at 45° C. to afford 12 (3.77 kg, 95%) as a mixture of monohydrates.

Melting point: 182° C.

Proton spectrum in methylene chloride d₂ at 250 MHz on Bruker DPX 250, with tetramethylsilane as internal reference. d=doublet, t=triplet, s=singlet, q=quartet. 8.66 ppm, d, 5.0 Hz, 1H; 8.34 ppm, dt, 1.6, 4.7 Hz, 1H; 7.85 ppm, dt, 1.6, 7.5 Hz, 1H; 7.68 ppm, dt, 1.2, 7.8 Hz, 1H; 7.43 ppm, m, 2H; 7.26 ppm, m, 1H; 7.23 ppm, bs, 1H; 6.98 ppm, s, 1H; 6.51 ppm, bt, 6.3 Hz, 1H; 4.88 ppm, s, 2H; 4.652 ppm, dd, 1.6, 4.7 Hz, 2H; 4.34 ppm, dt, 6.6, 14.4 Hz, 2H.

### 3-Fluoro-2-pyridylmethyl-3-(2,2-difluoro-2-(2-pyridyl)ethylamino)-6-chloropyrazin-2-one-1-acetamide Anhydrous A

A mixture of 12 (3.69 kg, 7.85 mol) and ethanol (140 L) was heated to reflux with a solution resulting when the internal temperature reached 78° C. The solution was allowed to cool to 67° C. when it was seeded with compound 12 anhydrous A (73 g, 0.15 mol). The resulting mixture was cooled to 40° C. and then 90 L of ethanol was removed by in vacuo distillation before allowing to cool to room temperature then stirring for 16 hours. The mixture was cooled to −5° C. then the product was collected by filtration and dried in vacuo at 50° C. to afford 3-fluoro-2-pyridylmethyl-3-(2,2-difluoro-2-(2-pyridyl)ethylamino)-6-chloropyrazin-2-one-1-acetamide anhydrous A (3.46 kg, 7.66 mol, 96% recovery).

### 3-Fluoro-2-pyridylmethyl-3-(2,2-difluoro-2-(2-pyridyl)ethylamino)-6-chloropyrazin-2-one-1-acetamide Monohydrate A

A mixture of 12 (20.0 g, 42.5 mmol) and aqueous ethanol (600 mL of 1:1 ethanol:water mixture) was heated to reflux during which dissolution occurred. The solution was cooled to 68° C. then 3-fluoro-2-pyridylmethyl-3-(2,2-difluoro-2-(2-pyridyl)ethylamino)-6-chloropyrazin-2-one-1-acetamide monohydrate A (0.05 g, 0.10 mmol) was added as seed. The resulting mixture was allowed to cool to room temperature over 1 hour and then cooled to 2° C. before the product was collected by filtration and dried under a stream of nitrogen at ambient temperature to afford the title compound (18.70 g, 93% recovery).

### 3-Fluoro-2-pyridylmethyl-3-(2,2-difluoro-2-(2-pyridyl)ethylamino)-6-chloropyrazin-2-one-1-acetamide Monohydrate B

A mixture of 12 (20.0 g, 42.5 mmol) and aqueous ethanol (400 mL of 4:1 ethanol:water mixture) was heated to 70° C. after which dissolution occurred. The solution was allowed to cool to room temperature over 16 hours then a further addition of water (320 mL) was made before the product was collected by filtration and dried under a stream of nitrogen at ambient temperature to afford the title compound (19.45 g, 97% recovery).

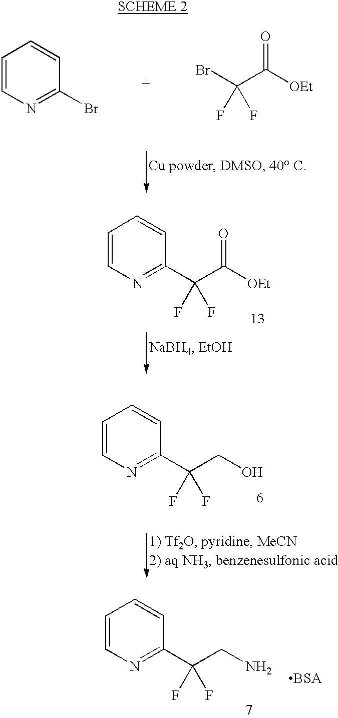

### Ethyl 2,2-difluoro-2-(2-pyridyl)acetate (13)

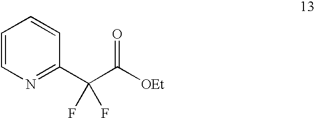

2-Bromopyridine (14.7 kg, 92.8 mol), ethyl 2-bromo-2,2-difluoroacetate (19.5 kg, 96.1 mol) and copper bronze (12.4 kg, 195 mol) were slurried in DMSO (74 L) and heated to 40° C. After 4 hours at this temperature, isopropyl acetate (119 L) was added and the reaction cooled to 5° C. A previously prepared, cooled (5° C.) solution of potassium dihydrogenphosphate (27.8 kg, 204 mol) in water (147 L) was added over 20 minutes, maintaining the temperature <15° C. The two-phase mixture was aged for 30 minutes then the aqueous layer was discarded. The organic extract was washed with water (59 L) and then distilled to residue under reduced pressure to give the title compound 13 (15.3 kg, 82% yield).

### Ethyl 2,2-difluoro-2-(2-pyridyl)acetate (13) (alternative process)

Copper powder (100.0 g) was slurried in DMF (500 ml) with 2-bromopyridine (100.0 g) and ethyl bromodifluoroacetate (133.3 g) and warmed to 50° C. The mixture was kept at between 50° C. and 52° C. for 8.5 hours, HPLC indicating no 2-bromopyridine remaining. The reaction mixture was diluted with isopropyl acetate (750 ml) and cooled to 5° C. A solution of potassium dihydrogen phosphate (190.7 g) in water (1100 ml) was added to the dimethylformamide/isopropyl acetate mixture at below 30° C. and well stirred for 30 minutes. The mixture was filtered to remove the precipitated copper salts giving a two phase filtrate. The solid was washed with isopropyl acetate (2×330 mL) and the washes combined with the filtrate. The layers were separated and the top organic layer washed twice with water (2×500 ml). Under reduced pressure the organic layer was evaporated to a volume of 300 mL, ethanol (400 mL) added, the volume reduced to 300 mL, again ethanol (400 mL) added, again the volume reduced to 300 mL then made up to 600 mL with ethanol. The required title compound 13 (103.15 g) was obtained in 81.0% yield.

### 2,2-Difluoro-2-(2-pyridyl)ethan-1-ol (6)

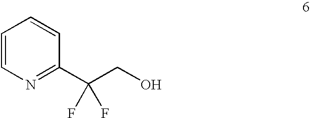

Ethyl 2,2-difluoro-2-(2-pyridyl)acetate (15.3 kg, 76.0 mol) (used as is from previous step) was dissolved in anhydrous ethanol (35 L) and filtered through a 1μ in-line filter into the vessel. Further alcohol (41 L) was used to rinse the lines. The batch was cooled to 5° C. and then sodium borohydride (2.88 kg, 76.0 mol) was added in 5 portions over 1 h, maintaining the temperature at <30° C. After the addition, the batch was aged for 1.5 h and then quenched by the careful addition of hydrochloric acid (55 L, 2 M aq sol) and then concentrated by vacuum distillation. The resulting aqueous solution was basified to pH 8.5 by the addition of sodium hydroxide solution (4.8 L, 46/48% w/w) and then extracted with ethyl acetate (56 L +28 L). The combined organic extracts were distilled to low volume (20 L) and then heptane (84 L) was added over 30 min, which induced crystallisation. The slurry was cooled to 5° C., aged for 1 h and then filtered. The filter cake was washed with heptane (5 L) and then dried overnight in vacuo at 30° C., to give the title compound 6 (11.7 kg, 76% yield over 2 steps from 2-bromopyridine) as crystalline cream plates.

Melting point: 58-60° C.; crystalline by XRPD

Proton NMR spectrum in methylene chloride d₂ at 400 MHZ on Bruker DRX400 spectrometer. s=singlet d=doublet, t=triplet, q=quartet, b=broad. 8.60 ppm dddt, 0.8,1.7,4.8 Hz, 1H; 7.89 ppm, dt, 1.7, 7.7 Hz, 1H; 7.71 ppm, dt, 1.2, 7.7 Hz, 1H; 7.44 ppm, ddq, 0.8, 1.2, 7.7 Hz, 1H; 4.19 ppm, t, 12.7, 2H; 3.65 ppm, bs, 1H.

### 2,2-Difluoro-2-(2-pyridyl)ethylamine.benzenesulfonate salt (7)

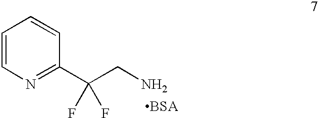

To a cooled (5° C.) solution of 2,2-difluoro-2-(pyridyl)ethanol 6 (20.0 g, 126 mmol), pyridine (13.2 mL, 163 mmol) and acetonitrile (200 mL) was added trifluoromethane sulfonic anhydride (39.0 g, 138 mmol) with the internal temperature kept below 18° C. The resulting mixture was stirred at 10° C. for 30 minutes and HPLC analysis indicated the reaction was complete.

Maintaining the temperature below 18° C., concentrated aqueous ammonia (s.g. 0.88, 200 mL) was added and the mixture was allowed to stir at room temperature for 16 hours. The orange solution was then evaporated in vacuo to a volume of approximately 200 mL. The dark aqueous solution was basified to pH14 with 48% sodium hydroxide solution (20 mL) and extracted with methylene chloride (2×200 mL). The methylene chloride extracts were combined and evaporated to a volume of 200 mL. A solution of benzenesulfonic acid (17.3 g; 108 mmol) in ethyl acetate (173 mL) was added over 1 hour at room temperature during which time crystallisation of the salt ensued. The mixture was cooled in ice for 1 hour and then the solid was filtered, washed with ethyl acetate (2×40 mL), dried in vacuo at 30° C. overnight to give the crystalline amine benzenesulfonate salt 7. (15.65 g, 79%). Methanol-d₄. Proton spectrum at 400 MHZ on Bruker DRX 400, with tetramethylsilane as internal reference. d=doublet, t=triplet, m=multiplet. 8.67 ppm, ddd, 0.9, 1.7, 5.9 Hz, 1H; 8.30 ppm, dt, 1.7, 7.7 Hz, 1H; 7.88-7.79 ppm, m, 3H; 7.59, dddd, 0.9, 2.0, 4.9, 7.7 Hz, 1H; 7.46-7.38 ppm, m, 3H; 3.91, t, 14.4 Hz, 2H.

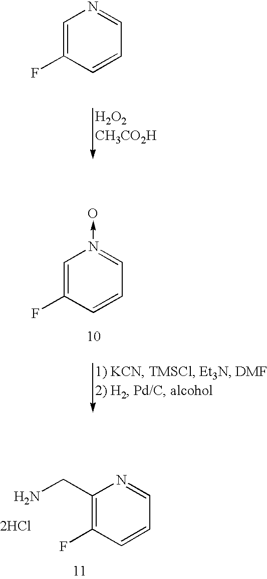

### 3-Fluoropyridine N-oxide (10)

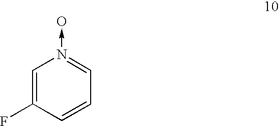

To a solution of 3-fluoropyridine (1.485 kg, 15.3 mol) in glacial acetic acid (12 L) at 100° C. was added 27% hydrogen peroxide solution (1.895 kg, 15.0 mol) dropwise over six hours. The solution was stirred overnight at the same temperature and the acetic acid/water then removed in vacuo. The residue was flushed in vacuo with toluene (6×8.4 L) before more toluene (12.2 L) was added followed by anhydrous potassium carbonate (1.023 kg, 7.4 mol). This mixture was stirred at 35° C. for 30 min and then filtered and the solids washed twice with warm (50° C.) toluene (2×4 L). The filtrate plus washes were concentrated in vacuo to 5.0 L and this was then heated to 45° C. and heptane (6.3 L) was added over 10 min. The slurry was cooled to 0° C. then aged for 2 hr before filtering. The solid was washed with heptane (1.3 L) and dried in vacuo at 30° C. to give the N-oxide 10 as a white solid (1.456 kg, 84%). Proton spectrum in methylene chloride d₂ at 250 MHz on Bruker DPRX 250. d=doublet, t=triplet, s=singlet, q=quartet. 8.03 ppm, dt, 1.8, 4.7 Hz, 1H; 7.92 ppm, dd, 0.7, 6.5 Hz, 1H; 7.15 ppm,br q, 8.2 Hz, 1H; 6.97 ppm, td, 2.2, 7.0 Hz, 1H.

### 2-Aminomethyl-3-fluoropyridine.2HCl (11)

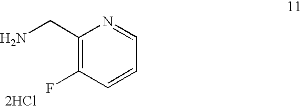

Chlorotrimethylsilane (7.19 kg, 66 mol) was added dropwise over ten minutes to a mixture of N-oxide 10 (2.5 kg, 22 mol), potassium cyanide (2.16 kg, 33 mol), triethylamine (6.7 kg, 66 mol) and DMF (30 L). The mixture was heated to 80° C. and held at this temperature for 24 hours after which a second charge of potassium cyanide (2.16 kg, 33 mol) was made and heating continued for a further 24 hours. The reaction was allowed to cool to room temperature overnight and then water (30 L) and IPAc (30 L) were added and the layers separated. The aqueous layer was re-extracted with IPAc (30 L) and the combined organics were then washed with 10% aq Na₂CO₃ (30 L) and brine (30 L). The combined organics were concentrated to a minimum volume (ca 7 L) and then denatured ethanol (14 L) was added. Concentration to a minimum volume (ca 7 L) afforded a solution of the crude nitrile (2.07 kg, 77% assay yield). In addition, this solution also contains 150 g of the regioisomeric nitrile.

Denatured ethanol (30 L) and conc HCl (4.1 L) were added to an ethanolic solution of the crude nitrile (4.2 kg, 2.07 kg assay, 17.0 mol). Damp 10% palladium on carbon (480 g, 0.1 dry weight equiv) was added and the mixture vigorously stirred under a hydrogen atmosphere (50 psi) for 6 hours. After degassing, the mixture was filtered and then activated carbon (Darco G-60, 480 g) was added and the mixture stirred for 1 hour at room temperature. After filtering to remove the carbon, the solution was concentrated to ca 14 L at which time precipitation of the amine.2HCl occurred. IPAc (20 L) was added dropwise over two hours with stirring and the solid was then isolated by filtration, dried (40° C. ) under vacuum, to afford 2-aminomethyl-3-fluoropyridine.2HCl 11 (2.37 kg, 70% yield, 98 LCAP). A second crop was obtained by concentrating the filtrate to 10 L and then addition of IPAc (10 L) followed by filtration of the crystalline 2-aminomethyl-3-fluoropyridine.2HCl (430 g, 13% yield, 90 LCAP).

### Recrystallization of 2-aminomethyl-3-fluoropyridine.2HCl (11)

2-Aminomethyl-3-fluoropyridine.2HCl 11 (2.0 kg, 10.0 mol, 98 LCAP) was suspended in acetic acid (20 L) under a nitrogen atmosphere with stirring and heated to 100° C. when complete dissolution had taken place. The solution was cooled slowly and precipitation started at 70° C. after which the HCl in acetic acid (5.0 L of 1.9 M sol) was added slowly. Cooling to room temperature took place overnight after which the solid was collected by filtration and washed with IPAc (15 L), then dried overnight at 40° C. in vacuo to afford the purified amine.2HCl (1.81 kg, 90% recovery) as a crystalline solid. Mp showed slow sublimation from 160° C. onwards. Proton spectrum in methylene chloride d₂ at 250 MHz on Bruker DRX 250. d=doublet, t=triplet, s=singlet, q=quartet. 8.40 ppm, dt, 1.2, 4.7 Hz, 1H; 7.60 ppm, dt, 1.2, 8.5 Hz, 1H; 7.41 ppm, br q, 4.3 Hz, 1H; 4.28 ppm, s, 2H.

What is claimed is:

1. A process for preparing 2,2-difluoro-2-(2-pyridyl)-ethan-1-ol, which comprises coupling 2-bromopyridine with ethyl 2-bromo-2,2-difluoroacetate, in the presence of metallic copper and a solvent, to form 2,2-difluoro-2-(2-pyridyl)acetate.

2. A process of claim 1, wherein the metallic copper is nonactive copper powder, and the solvent is a non-reactive solvent, the reducing agent is a borohydride or aluminium hydride reagent.

3. A process of claim 2, wherein the nonactive copper powder is copper-bronze, the solvent is dimethyl sulfoxide or dimethylformamide, and the reducing agent is selected from the group consisting of sodium borohydride, lithium borohydride, sodium aluminium hydride, and lithium aluminium hydride.

4. A process for preparing 2,2-difluoro-2-(2-pyridyl)ethylamine benzenesulfonate salt which comprises adding trifluoromethanesulfonic anhydride to a solution of 2,2-difluoro-2-(pyridyl)ethanol in acetonitrile, and thereafter adding an ammonium reagent and a solution of benzenesulfonic acid to form 2,2-difluoro-2-(2-pyridyl)ethylamine benzenesulfonate salt.

5. A process of claim 4, wherein the ammonium reagent is aqueous ammonia.

6. A process for preparing ethyl 2-{(N-(2,2-dimethoxyethyl)carbamoyl}carbonylamino)acetate which comprises reacting ethyl glycine hydrochloric acid salt with ethyl oxalyl chloride in the presence of a first non-reactive solvent to form a reaction product, adding 2,2-dimethoxyethylamine to the reaction product to form ethyl 2-{(N-(2,2-dimethoxyethyl)carbamoyl}carbonylamino)acetate, and precipitating the ethyl 2-{(N-(2,2-dimethoxyethyl)carbamoyl}carbonylamino)acetate with a second non-reactive solvent.

7. A process of claim 8, wherein the first non-reactive solvent is isopropyl acetate and the second non-reactive solvent is heptane.

8. A process for preparing ethyl 3-(2,2-difluoro-2-(2-pyridylethylamino)pyrazin(1H)-2-one-1-acetate which comprises coupling ethyl 3-chloropyrazin(1H)-2-one-1-acetate with 2,2-difluoro-2-(2-pyridyl)ethylamine benzenesulfonate salt in the presence of a base, a non-reactive solvent, and an iodide, to form ethyl 3-(2,2-difluoro-2-(2-pyridylethylamino)pyrazin(1H)-2-one-1-acetate.

9. A process of claim 8, wherein the iodide is sodium iodide.

10. A process of claim 8, wherein the base is N,N-diisopropylethylamine, triethyl amine or tributyl amine, and the non-reactive solvent is acetonitrile

11. A process for preparing 3-fluoro-2-pyridylmethyl-3-(2,2-difluoro-2-(2-pyridyl)ethylamino)-6-chloropyrazin-2-one-1-acetamide which comprises coupling 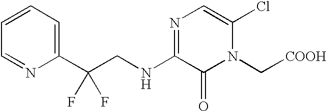
with 2-aminomethyl-3-fluoropyridine dihydrochloric acid salt in the presence of a coupling reagent and a base to form 3-fluoro-2-pyridylmethyl-3-(2,2-difluoro-2-(2-pyridyl)ethylamino)-6-chloropyrazin-2-one-1-acetamide.

12. A process of claim 11, wherein the coupling reagent is selected from 1-(3-dimethylaminopropyl)-3-ethylcarbdiimide hydrochloride and 1,3-dicyclocarbdiimide.

13. A process of claim 11, wherein the base is potassium hydroxide.

14. A compound having the formula 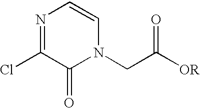
wherein R is C₁₋₄ alkyl.

15. A compound of claim 14 wherein R is —CH₂CH₃.

16. A process for recrystallizing 2-aminomethyl-3-fluoropyridine dihydrochloride which comprises suspending 2-aminomethyl-3-fluoropyridine dihydrochloride in acetic acid, heating to dissolve the 2-aminomethyl-3-fluoropyridine dihydrochloride into a solution, and precipitating the 2-aminomethyl-3-fluoropyridine dihydrochloride from the solution.

17. A process for preparing 2-cyano-3-fluoropyridine comprising adding chlorotrimethylsilane to a mixture comprising 3-fluoropyridine N-oxide and potassium cyanide to form 2-cyano-3-fluoropyridine.

18. A process for preparing ethyl 2-(2,3-dioxo-1,4-dihydropyrazinyl)acetate which comprises preparing ethyl 2-{(N-(2,2-dimethoxyethyl)carbamoyl}carbonylamino)acetate by reacting ethyl glycine hydrochloric acid salt with ethyl oxalyl chloride in the presence of a first non-reactive solvent to form a first reaction product, adding 2,2-dimethoxyethylamine to the first reaction product to form a second reaction product comprising ethyl 2-{(N-(2,2-dimethoxyethyl)carbamoyl}carbonylamino)acetate, and cyclizing ethyl 2-{(N-(2,2-dimethoxyethyl)carbamoyl}carbonylamino)acetate in the presence of an acid reagent and a second solvent to form ethyl 2-(2,3-dioxo-1,4-dihydropyrazinyl)acetate.

19. A process of claim 18, wherein the first non-reactive solvent is isopropyl acetate, the acid reagent is trifluoroacetic acid and the second solvent is acetic acid.
